(19) United States
(12) Patent Application Publication
(10) Pub. No.: US 2020/0194099 A1
(43) Pub. Date: Jun. 18, 2020

(54) MACHINE LEARNING-BASED VARIANT CALLING USING SEQUENCING DATA COLLECTED FROM DIFFERENT SUBJECTS

(71) Applicant: ACCURASCIENCE LLC, Johnston, IA (US)

(72) Inventors: Tongbin Li, Johnston, IA (US); Yuhao Chen, Johnston, IA (US); Zhishuang Li, Johnston, IA (US)

(21) Appl. No.: 16/679,101

(22) Filed: Nov. 8, 2019

Related U.S. Application Data
(63) Continuation-in-part of application No. 15/953,087, filed on Apr. 13, 2018, now Pat. No. 10,600,501.
Application No. 15/953,087 is a continuation of application No. 14/358,643, filed on May 15, 2014, now Pat. No. 10,089,436.
(60) Provisional application No. 61/898,680, filed on Nov. 1, 2013.

(51) Int. Cl. G16B 30/00; G16B 40/00; G16B 20/00

(52) U.S. Cl. CPC G16B 30/00; G16B 20/00; G16B 40/00

(57) ABSTRACT

An example computer-implemented genotyping method includes identifying a locus of interest from a reference sequence; and obtaining sequencing data collected from a target subject. The sequencing data collected from the target subject includes a plurality of sequencing reads collected from the target subject using a hardware sequencing machine. The method further includes: aligning each sequencing read in the plurality of sequencing reads collected from the target subject based on the locus of interest identified from the reference sequence to produce a plurality of aligned sequencing reads; obtaining genotype data from a plurality of baseline subjects, wherein the plurality of baseline subjects are subjects different from the target subject; and identifying a genotype for the target subject at the locus of interest identified from the reference sequence based on (1) the plurality of aligned sequencing reads and (2) the genotype data obtained from a plurality of baseline subjects.

### RELATED APPLICAITONS

This application is a continuation in part of U.S. patent application Ser. No. 15/953,087, filed Apr. 3, 2018, which is a continuation of U.S. patent application Ser. No. 14/358,643, filed May 15, 2014, issued as U.S. Patent 10,089,436, on Oct. 2, 2018, which is a U.S. national stage patent application under 35 U.S.C. §371 of PCT/CN2014/072043, filed Feb. 13, 2014, which claims the benefit of U.S. provisional patent application Ser. No. 61/898,680, filed Nov. 1, 2013.

All above-identified patent and patent applications are hereby incorporated by reference in its entirety.

### TECHNICAL FIELD

The present disclosure relates generally to variant calling and more particularly to machine learning-based variant calling in accordance with sequencing data collected from a threshold number of different subjects.

### BACKGROUND

High-throughput sequencing technologies (such as those developed by Illumina, Ion Torrent, Pacific Biosciences and Oxford Nanopore) offer avenues for uncovering genetic variations in individuals' genomes and for analyzing genetic factors underlying common and rare diseases.

These technologies have, however, limited applications, due to their high error rates intrinsic in the data generated through these technologies. The reported error rates in these data are approximately between 0.5% and 5%, which are orders of magnitude higher than the error rates presented in the older, low-throughput sequencing platform often referred to as the “Sanger sequencing” technology (whose error rates is approximately between 0.0001% and 0.01%).

These high error rates pose a great technical challenge to the bioinformatics process known as “variant calling” or “genotyping,” which often require a much lower error rate in order to produce meaningful variant calling results.

The above identified technical problems are reduced or eliminated by the systems and methods disclosed in this disclosure.

### BRIEF DESCRIPTION OF THE DRAWINGS

FIG. 1 is a block diagram illustrating an example deep sequencing method process in accordance with some implementations.

FIG. 2 is a block diagram illustrating example correlations in the genotypes between varied loci at adjacency in accordance with some implementations.

FIG. 3 is a fist table illustrating example comparisons of variant calling accuracies achieved using a SVM-based variant calling method, and single-sample and multi-sample GATK variant calling methods in accordance with some implementations.

FIG. 4 is a block diagram illustrating an example class 3 variant calling method in accordance with some implementations.

FIG. 5 is a second table illustrating example comparisons of variant calling accuracies achieved using GATK, SVM-based, and CNN-based variant calling methods in accordance with some implementations.

FIG. 6 is an example block diagram illustrating example genotype data 600 which may be used in a machine learning -based variant calling for in accordance with some implementations.

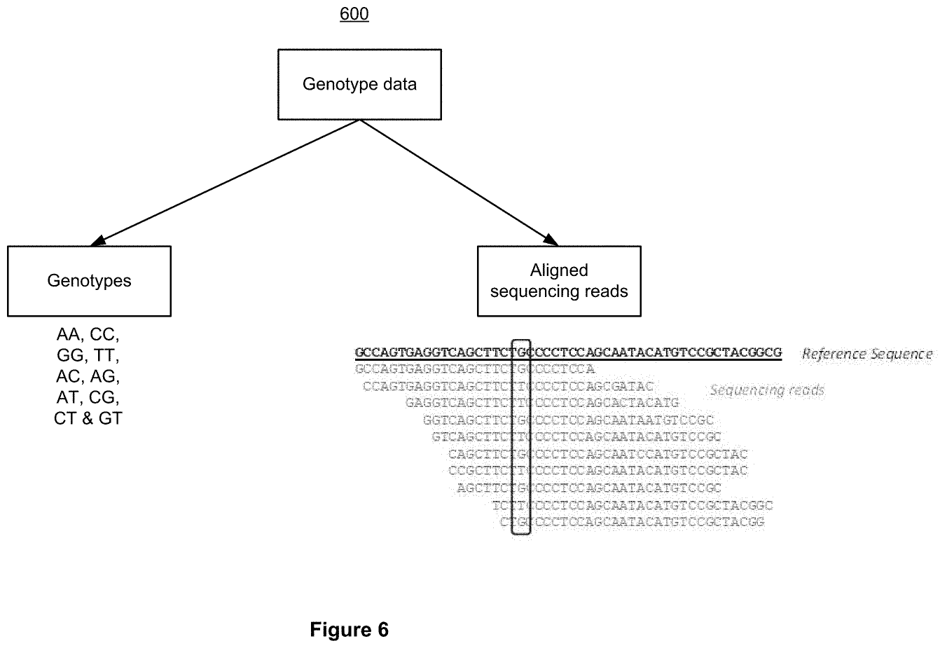

FIG. 7 is an example block diagram illustrating an example method 700 for machine learning-based variant calling in accordance with some implementations.

FIG. 8 is an example block diagram illustrating an example method for machine learning-based variant calling in accordance with some implementations.

FIG. 9 is an example block diagram illustrating an example computer system for machine learning-based variant calling in accordance with some implementations.

The implementations disclosed herein are illustrated by way of example, and not by way of limitation, in the figures of the accompanying drawings. Like reference numerals refer to corresponding parts throughout the drawings.

### SUMMARY

Technologies relating to machine learning-based variant calling (or genotyping) in accordance with sequencing data collected from a threshold number of different subjects are disclosed.

An example computer-implemented genotyping method includes identifying a locus of interest from a reference sequence; and obtaining sequencing data collected from a target subject. The sequencing data collected from the target subject includes a plurality of sequencing reads collected from the target subject using a hardware sequencing machine. The method further includes: aligning each sequencing read in the plurality of sequencing reads collected from the target subject based on the locus of interest identified from the reference sequence to produce a plurality of aligned sequencing reads; obtaining genotype data from a plurality of baseline subjects, wherein the plurality of baseline subjects are subjects different from the target subject; and identifying a genotype for the target subject at the locus of interest identified from the reference sequence based on (1) the plurality of aligned sequencing reads and (2) the genotype data obtained from a plurality of baseline subjects.

Identifying the genotype for the target subject at the locus of interest identified from the reference sequence is, in some implementations, further based on (3) genotype data determined for one or more loci other than the locus of interest for the target subject.

The genotype data includes, in some implementations, one or more genotypes determined for one or more loci for the plurality of baseline subjects.

The one or more loci includes, in some implementations, the locus of interest. The one or more loci, in some implementations, excludes the locus of interest. The one or more loci, in some implementations, includes the locus of interest and at least one locus other than the locus of interest.

The genotype data includes, in some implementations, a second plurality of aligned sequencing reads collected from the plurality of baseline subjects.

The method includes, in some implementations, aligning each sequencing read in the second plurality of sequencing reads collected from the baseline subjects based on the locus of interest identified from the reference sequence to produce a second plurality of aligned sequencing reads collected from the baseline subjects.

The aligned sequencing reads of the plurality of baseline subjects include, in some implementations, sequencing reads including the locus of interest and one or more loci other than the locus of interest.

The one or more loci other than the locus of interest include, in some implementations, varied loci that are within a predefined distance from the locus of interest.

The varied loci include, in some implementations, loci with minor allele frequency greater than a predetermined threshold within the plurality of baseline subjects.

The aligned sequencing reads of the plurality of baseline subjects included, in some implementations, sequencing reads that do not include the locus of interest. The aligned sequencing reads of the plurality of baseline subjects, in some implementations, include sequencing reads that include only the locus of interest.

The method includes, in some implementations, identifying the genotype for the target subject at the locus of interest identified from the reference sequence includes applying a machine learning-based classification algorithm. The machine learning-based classification algorithm is trained in accordance with samples with known genotypes.

The classification method used by the machine learning-based classification algorithm is, in some implementations, selected from one of: a Naive Baise (NB) classification method, a Logistic Regression (LR) classification method, a Support Vector Machine (SVM) classification method, and a Random Forest (RF) classification method.

The machine learning-based classification algorithm uses, in some implementations, a classification method selected from one of: a deep fully-connected Artificial Neural Network (ANN) classification method, a Convolutional Neural Network (CNN) classification method, a Recurrent Neural Network (RNN) classification method, or a classification method derived from a network structure derived from any of the preceding network structures.

The hardware sequencing machine is, in some implementations, a next-generation sequencing equipment. The next-generation sequencing equipment, in some implementations, is an Illumina, Ion Torrent, Pacific Biosciences or Oxford Nanopore sequencing machine. The next-generation sequencing equipment, in some implementations, is a high throughput sequencing machine.

The method is, in some implementations, executed by a computer that is different and separate from the hardware sequencing machine.

The target subject and the plurality of baseline subjects are, in some implementations, human subjects.

Identifying the genotype for the target subject at the locus of interest identified from the reference sequence is, in some implementations, further based on (3) genotype data determined for one or more loci other than the locus of interest for the target subject.

### DETAILED DESCRIPTION

Variant calling often refers to the determination of whether a genetic variant has occurred at a particular locus (a genetic variant could be a Single-Nucleotide Variation (SNV), short insertion, deletion/InDel, or Structural Variation (SV)). Genotyping refers to the determination of the precise genotype at a given locus. For human and other diploid species, there are 10 possible diploid genotypes at each genomic locus: AA, CC, GG, TT, AC, AG, AT, CG, CT, and GT). In the present disclosure, the terms “variant calling” and “genotyping” may be used interchangeably. The present disclosure treats the SNVs as an example or representative variant type, with the understanding that the same principles discussed in this disclosure apply equally to the calling of other variants of other types (for example, InDels and SVs).

One of the reasons variant calling is technically challenging when handling high-throughput sequencing data having high error rates is after the sequencing data are aligned to the reference sequence (e.g., the human reference genome), both sequencing errors (which are also referred to as sequencing noise or noise for short)and true genetic variants may be identified as differences between the sequencing data and the reference. When sequencing errors are identified as differences between the sequencing data and the reference, an incorrect variant calling is made. Incorrect variant callings are to be eliminated or reduced to improve accuracy.

Sequencing noise occurs at even higher rates (for example, between 0.5% and 5%) than the “signal” or true genetic variants (e.g., SNVs occur at a rate of approximately 0.1%), making it technically difficult to achieve accurate calling of variants.

Class 1 Variant Calling Methods

One way to address this challenge is through “deep sequencing”, which refers to the practice of acquiring sequencing data repeatedly at a high level of redundancy or depths.

The redundancy or depth of a sequencing experiment refers to the total number of times the locus of interest in the genome is sequenced. For example, if a locus of interest has been sequenced 10 times, or, as is commonly narrated, the locus is covered by 10 sequencing reads (a sequencing read is a small unit of a sequencing experiment; a sequencing experiment may include tens of millions of sequencing reads), then the sequencing depth at the locus of interest is 10.

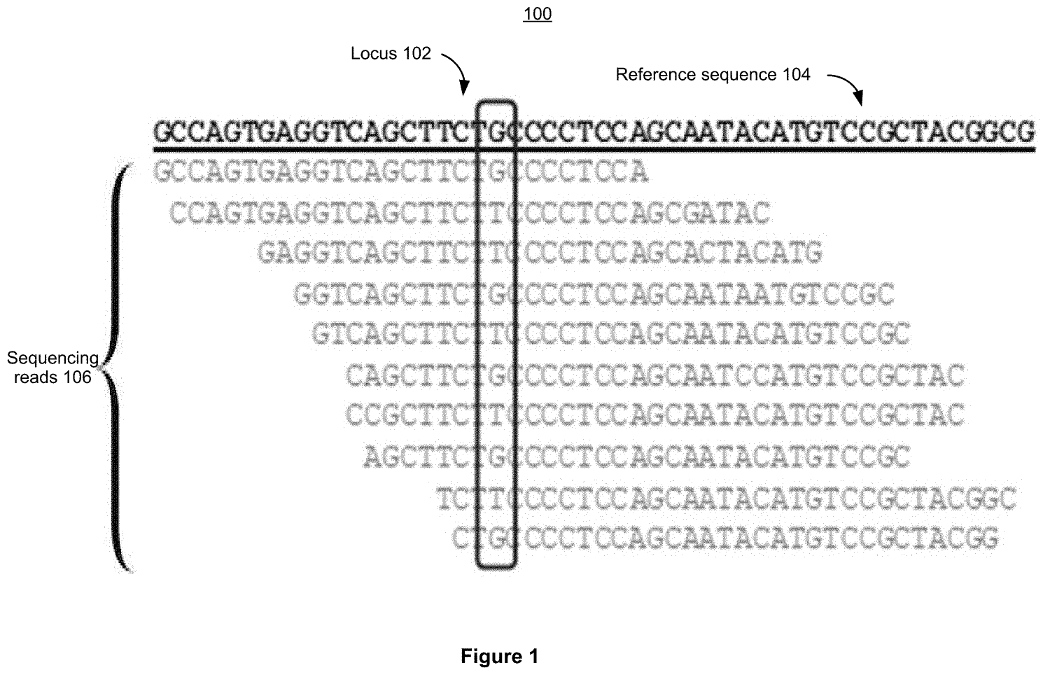

FIG. 1 is a block diagram illustrating an example deep sequencing method process 100 in accordance with some implementations.

As shown in FIG. 1, the reference sequence 104 refers to a Reference Genome. The reference sequence 104, in some implementation is a human reference genome, such as the GRCh38 released by the Genome Reference Consortium in 2013.

As shown in FIG. 1, Sequencing reads 106 include 10 reads, each of which includes the locus 102. In other words, the locus 102 has been sequenced 10 times, or, as is commonly narrated, the locus is covered by 10 sequencing reads (a sequencing read is a small unit of a sequencing experiment; a sequencing experiment may include tens of millions of sequencing reads), then the sequencing depth at the locus 102 is 10.

Before variant calling/genotyping, sequencing reads need to be aligned to proper regions or loci at the reference sequence. This is a bioinformatically challenging process considering the large number of sequencing reads (sometimes hundreds of millions), the large size of the reference sequence (about 3 billion positions in the case of the human reference genome) and the fact that the reference sequence often includes repeated subsequences. However, the sequencing read alignment algorithms are not a focus of the current disclosure. The technologies described in this disclosure may be applied when sequencing reads are properly aligned.

One of the working assumptions of deep sequencing is, while true genetic variants always occur at the same loci in a genome, sequencing errors often occur randomly across sequencing reads. Thus, if the same locus is repeatedly sequenced through a sufficient number of sequencing reads, then intelligent bioinformatics methods (which may also be referred to as variant calling methods) can distinguish between sequencing errors and true genetic variants based on the different patterns in which they occur.

Many variant calling or genotyping methods (for example, the GATK and SOAPsnp methods) are Bayesian inference-based statistical methods. A Bayeian inference-based variant calling method defines a prior probability for each possible genotype before the sequencing data are observed, then at the observation of the sequencing data; the Bayeian inference-based variant calling method calculates the conditional probability or likelihood of observing the sequencing data condition on the genotype.

Next, a posterior probability is calculated for each genotype as the product of the prior probability and the conditional probability. The genotype with the highest posterior probability is called for the locus. GATK and SOAPsnp are considered class 1 variant calling methods (or class 1 variant callers). When a class 1 variant caller attempts to call variants at a particular genomic locus for a target subject, the following may be taken into consideration:(1) the sequencing data (including quality information of the sequencing data), (2) the reference sequence and (3) other information relating directly to the locus of interest. Sequencing data or other information at other loci of the genome are, however, not considered as part of a class 1 variant calling process.

Class 2 Variant Calling Methods

When a class 2 variant caller attempts to call variants at a particular locus for a target subject, it not only takes into consideration (1) the sequencing data and (2) other information directly related to the locus of interest, but also (3) the sequencing data and (4) other information obtained from other genomic loci of the subject. In these ways, class 2 variant callers may achieve higher variant calling accuracies than class 1 variant callers. Example class 2 variant callers include PMCaller and DeepVariant. When PMCaller method is used, parameter values in a SNV table and error tables are calculated based on sequencing data from other genomic loci of the subject, and are used in the variant calling of the particular locus of interest for the target subject. DeepVariant is a deep artificial neural network (ANN)-based machine learning method where parameters of the deep ANN are fixed through training using sequencing data and other information from other genomic loci of the target subject, then the deep ANN is used to conduct variant calling at the locus of the interest.

PMCaller is a Bayesian inference-based method. It embodies a SNV table which models the prior probabilities of occurrences of all possible variants defined as combinations of a reference base and a diploid genotype (i.e., A->AA, A->AC, A->AG, A->AT, . . . T->TT), and error tables which model the conditional probabilities of occurrences of all possible sequencing errors defined as combinations of a diploid genotype and a base call (i.e., AA->A, AA->C, AA->G, AA->T, . . . , TT->T).

The values in the SNV table and error tables are calculated based on the sequencing data of the higher depth or higher confidence regions of the genome, and are then used in the variant calling of lower depth or lower confidence loci. In these ways, PMCaller “borrows the power” of the sequencing data that cover other, unrelated regions of the genome, when making variant calling of a locus of interest. Benchmark shows that at average depth of 15-20, PMCaller produces variant calling error rates that are 30-100 times lower than those produced by class 1 variant callers such as GATK and SOAPsnp.

DeepVariant is another class 2 variant calling method. Instead of adopting the Bayesian inference strategy, DeepVariant applies a machine learning approach. It treats a genomic locus as a sample, and assembles information of a 201-base window—including the base of interest and two 100-base regions at each direction surrounding the base of interest (for example, 100 bases preceding the base of interest and another 100 bases succeeding the base of interest).

The data required for a DeepVariant method includes (1) the reference sequence, (2) identity of each base in all sequencing reads aligned to the 201-base window, (3) quality score associated with each base in the sequencing data (generated together with the sequencing data by the sequencing instrument), and (4) other information such as information related to alignment quality. DeepVariant converts and represents date items (1)-(4) into an image format, and uses the image as an input to Inception, a deep Convolutional Neural Network (CNN) model, to approximate the relationship between the input information and variant calling results. Similar to other machine learning methods, the Inception model is established through training, using a rich set of samples with known genotypes.

Following training, the performance of an Inception model may be evaluated using testing datasets. Like PMCaller, DeepVariant is a class 2 variant caller, because it is established through training using sequencing data and other information that cover other loci of the genome of the same subject, unrelated to the locus of interest. Test showed that DeepVariant's variant calling error rates were 10-100 times lower than GATK, a class 1 variant caller.

The classes 1 and 2 variant calling methods make variant calling using one target subject's sequencing data at a time. With the ever decreasing cost of acquiring high-throughput sequencing data, and a number of sequencing projects of ever increasing scales (e.g., the 1000 Genomes Project in the United States the 100,000 Genomes Project in the United Kingdom, and BGI Group's plan to sequence 1,000,000 human subjects), sequencing data for increasingly large populations have become available.

Class 3 Variant Calling Methods

In view of the increasingly available sequencing data collected from a large number of subjects (human, plants, animal, or any other subjects), variant calling methods that effectively take advantage of multiple subjects'sequencing data—which may be referred to as class 3 variant calling methods—can perform substantially better than class 1 and class 2 variant calling methods.

Class 3 variant calling methods can “borrow the power” from the sequencing data and other knowledge extracted from not only (1) other loci of the genome from the same subject, but also (2) the same locus of the genome from multiple different subjects. Class 3 variant calling methods have the potential of achieving substantially better performance than class 1 and class 2 variant callers, because they can “borrow the power” from the sequencing data and other knowledge extracted from not only other loci of the genome from the same subject, but also from the same locus as well as other loci of the genome from multiple other subjects.

GATK embodies a multiple sample variant calling method within its Bayesian inference-based framework. However, it considers sequencing data and related information at the loci of interest only, and does not “borrow the power” from information from other loci of the genome. Test suggests that GATK's multi-sample variant calling method offers some advantage over single-sample variant calling at very lower depths (<2), but it performs very poorly and much worse than the single-sample GATK method. GATK is thus considered a class 1 variant caller. Benchmark data show that the machine learning-based multi-subject calling method described in the present disclosure propose substantially out-performs GATK's Bayesian inference-based method under the multi-subject setting even when only the information related directly to the loci of interest is considered.

The idea of making use of the relationships of variants occurred at multiple loci across multiple subjects is conceptually related to the imputation methods adopted in microarray-based genotyping practice. Some important contributions in this field include methods and implementations such as MACH, Beagle and IMPUTE2 However, those methods were developed and applied in a fundamentally different domain practice: they were to “impute” the genotype when there were no measurement data available for the loci of interest, rather than to improve the variant calling accuracy as in the current application. Moreover, the statistical methods applied in those imputation methods (most of which are hidden Markov Model (HMM)-based) are fundamentally different from the machine learning-based methods described in the present disclosure.

Population genetics theories suggest that genotypes at adjacent genomic loci are likely correlated due to linkage disequilibrium. Next, it is established that such correlation is sufficiently strong to offer benefit in the class 3 or multi-subject, multi-locus variant calling setting. Several multi-subject exome and whole-genome sequencing datasets (each consisting of >200 subjects) are analyzed, and focus are given on a subset of genomic loci with minor allele frequency >2% across all subjects in a dataset. These loci are termed “varied loci”. It is estimated that on average, varied loci occur once every ˜150 bases in the coding regions, and once every ˜390 bases in the non-coding regions of the human genome. The minor allele frequency cut-off could be set at a lower level for a dataset of a larger subject number, resulting in more densely distributed varied loci to be counted. For instance, for a future dataset that includes 1,000,000 subjects, a minor allele frequency cut-off could be set at 0.01% level or even lower, leading to much larger numbers of varied loci to be identified in the genome. Therefore, although the current technologies focus on genomic loci with moderate chance to carry variants, these technologies can be extended to loci for which extremely rare (or private) variants may occur.

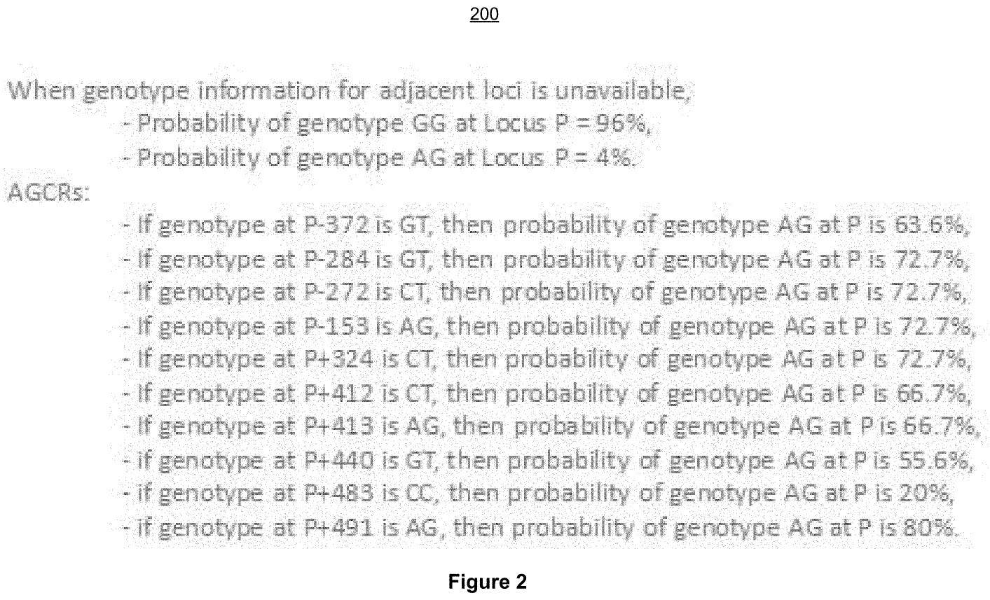

What has been shown in evidence of strong correlation in the genotypes between varied loci at adjacency, which could potentially be used to improve the variant calling of a varied locus. Correlations that could potentially be beneficial for the variant calling of a varied locus of interest (which is designated as “locus P”) may be summarized in the form of a set of probability rules, which may be referred to as adjacent genotype correlation rules, or AGCRs. For instance, for a locus P at chr. 1, position 13246276 at human reference genome version hg38/GRCh38 (see for example FIG. 2), if no information regarding the genotypes of any of its adjacent loci is available, there is 96% probability for locus P to have the genotype GG, and 4% probability for it to have the genotype AG. FIG. 2 is a block diagram illustrating example correlations 200 in the genotypes between varied loci at adjacency in accordance with some implementations.

However, if the genotype of the varied locus at position P-372 (i.e., position 13245904) is known to be GT, then the probability for locus P to have the genotype AG increases sharply to 63.6%. If the genotype of the varied locus at P+324 (i.e., position 13246228) is known to be CT, then the probability for locus P to have the genotype AG increases to 72.7%. Within the 1001-position window centering around this particular locus P (including 500 positions to each direction), there are a total of 10 varied loci for which AGCRs are derived which lead to the probability of locus P to have the genotype AG to be >15%.

Similar AGCRs may be derived for ˜44% of all varied loci, and for these varied loci for which AGCRs could be derived, on average 7.9 AGCRs are present for each varied locus. The existence of these AGCRs is strong evidence that there is substantial correlation among genotypes of adjacent genomic loci, and such correlation could be harvested to achieve improved variant calling under the class 3 (i.e. multi-subject, multi-locus) setting. The benefit of such correlation in variant calling is expected to be substantially amplified with increasing availability of sequencing datasets of much larger subject numbers (e.g., >100,000).

Machine Learning-Based Variant Calling Methods

One way by which the correlation in genotypes of adjacent genomic loci could be used in variant calling is to incorporate the AGCRs into the prior probabilities under a conventional Bayesian inference-based variant calling framework.

Another, more flexible way to make use of such correlation information is to construct the variant calling method using a machine learning-based framework, include the information related to the genotypes—and the sequencing data—related to the adjacent loci as input to a classification model, and establish the model through a learning process.

SVM-Based Variant Callers

In accordance with dataset obtained from the dbGAP database that includes exome sequencing data of 1263 subjects, the performance of machine learning-based variant calling methods are evaluated. Genomic loci that meet a set of predefined criteria which allow confident genotyping to be confidently made using a “naive caller” are focused on first. These predefined criteria may include: depth is ≥50, and the ratio of read number of the second most popular base is either ≤0.15 (for which the naive caller will call a homozygous genotype) or between 0.45 and 0.5 (for which the naive caller calls a heterozygous genotype). Then, a resampling routine was performed, which may be termed “shallowing”, where a fixed proportion of the sequencing reads were taken randomly without replacement. For example, if the average depth of an original dataset (before shallowing) is 60, then a shallowing routine at a 1:10 ratio will generate a dataset of average depth of 6. These data following shallowing were provided to a variant calling method as input. The performance of the variant calling was evaluated, with the genotyping results called before shallowing by the naive caller as ground truth.

In an example SVM-based variant caller, input variables (or features) may include: the reference base at the locus, the depth (or number of reads) covering the locus (which may be referred to as locus P), and the proportions of reads for each of the 4 bases (A, C, G and T). Also included are reference base, the depth, and proportions of reads for each of the 4 bases at each of varied loci within a 1001-position window centering around locus P. Other features include the variant calling results made by the naive caller for each of the varied loci. If a varied locus—following shallowing—met the criteria for the naive caller to make a confident genotyping call (i.e., depth is ≥50, and the ratio of read number of the second most popular base is either ≤0.15 or between 0.4 and 0.5), then this variant calling result is used. Otherwise, a dummy value is used to represent “not available” for this feature. The radial basis function (RBF) kernel is used in the SVM model. The recursive feature elimination (RFE) feature selection routine may be adopted to select the most informative features. Data for 421 subjects are randomly selected and reserved them as testing set. The remaining 842 subjects are randomly split into a 672-subject training set and a 170-subject validation set. The parameter values for SVM model (gamma and C) and number of features retained in the RFE routine were tuned based on the model performance in the validation set. The final model performance was evaluated using the testing set.

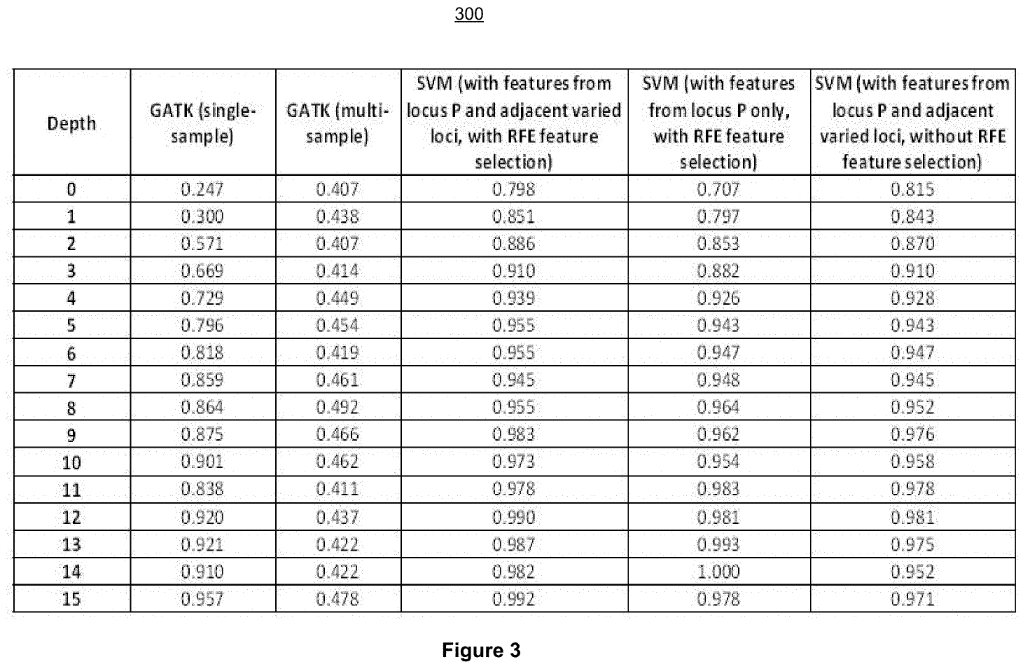

FIG. 3 is a fist table 300 illustrating example comparisons of variant calling accuracies achieved using a SVM-based variant calling method, and single-sample and multi-sample GATK variant calling methods in accordance with some implementations.

FIG. 3 summarizes the performance of the SVM-based variant caller under a few different settings and how it compares with GATK (single-sample and multi-sample settings), at a series of sequencing depths—between 0 and 15. Evaluating variant calling performance at these relatively lower sequencing depths may be key to measure a SVM-based variant caller's performance, because these depths represent most challenging situations where class 1 and class 2 variant callers have substantial difficulties achieving accurate calling results.

FIG. 3 shows that SVM-based variant caller consistently out-performs GATK by very substantial margins. For example, at depth=5, whereas GATK (single-sample setting) produces an accuracy of 0.796, the SVM-based variant caller (with RFE feature selection) achieves an accuracy of 0.955. At a higher depth of 10, when GATK (single-sample setting) achieves an improved accuracy of 0.901, the SVM-based variant caller (with RFE feature selection) demonstrates an even higher accuracy of 0.973.

FIG. 3 also shows that whereas multi-sample GATK achieved improved performance over single-sample GATK at very low depth (between 0 and 1), its advantages diminishes as sequencing depth reaches 2 or above. In fact, its accuracy persists at a level of 0.40-0.49 with increasing depths, when single-sample GATK achieves a much higher accuracy (>0.9) when the depth reaches 10 or higher, suggesting that multi-sample GATK does not make effective use of multiple subjects' information, thus GATK should only be treated as a class 1 variant caller.

In contrast, the SVM-based variant caller that only considers information related to locus P—but not adjacent varied loci's information, performs well: at depth=5, it produces an accuracy of 0.943, only slightly lower than that of the SVM-based caller that considers both locus P and adjacent varied loci's information.

Feature selection routine RFE may produce improvement to the performance of SVM-based variant caller. For example, at depth=5, the SVM-based variant caller without RFE achieves an accuracy of 0.943, slightly lower than the accuracy 0.955 achieved by the caller with RFE. At depth=10, the caller without RFE achieves an accuracy of 0.958, as compared to the accuracy 0.973 achieved by the caller with RFE.

Deep Artificial Neural Network-Based Variant Callers

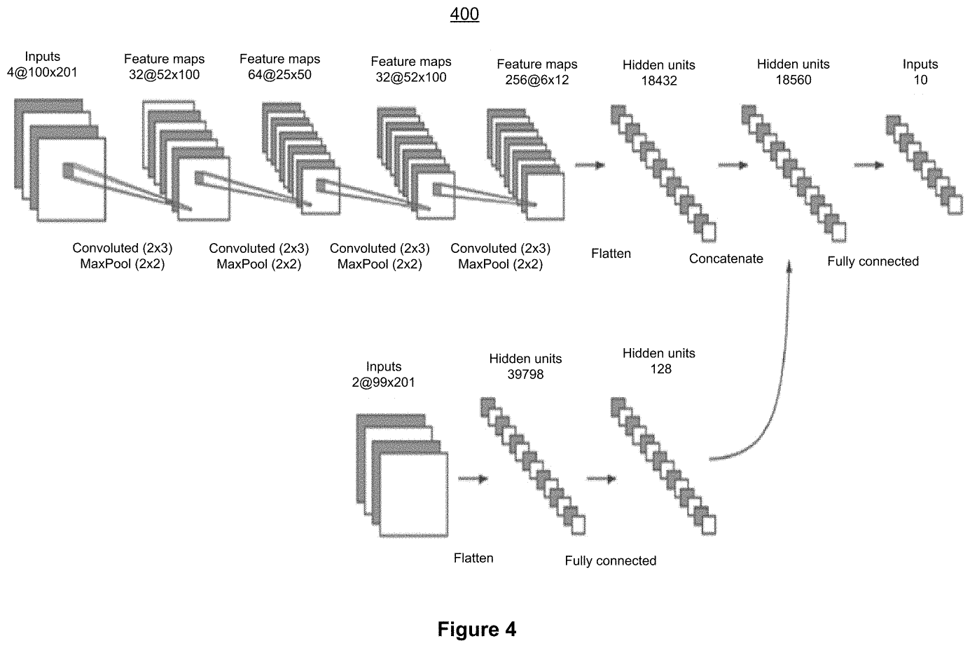

Deep ANN-based class 3 variant callers have also been developed, using several basic ANN structures including fully connected ANNs, convolutional neural networks (CNNs) and recurrent neural networks (RNNs). An example CNN-based variant caller is shown in FIG. 4, which is a block diagram illustrating an example class 3 variant calling method 400 in accordance with some implementations.

The example CNN-based variant caller shown in FIG. 4 includes two horizontal paths. The upper path is an approximation of the DeepVariant model, which processes input related to Locus P, whereas the lower path processes input related to varied loci within a 1001-position window centering around this particular locus P—just as in the SVM-based variant caller, as well as Locus P ID and subject ID. The upper path contains 4 convolution layers each followed by a MaxPooling layer, a flattening and a concatenation operation then followed by a fully connected hidden layer, which is in turn connected to an output layer representing the 10 diploid genotypes. The lower path includes a flattening operation followed by a fully connected hidden layer, and it joins the upper path at the input of its fully connected hidden layer.

Data following the same shallowing procedure as with the SVM-based variant caller are prepared and then split the data into a training set (e.g., with 613915 samples), a validation set (e.g., with 237460 samples), and a testing set (e.g., with 13511 samples).

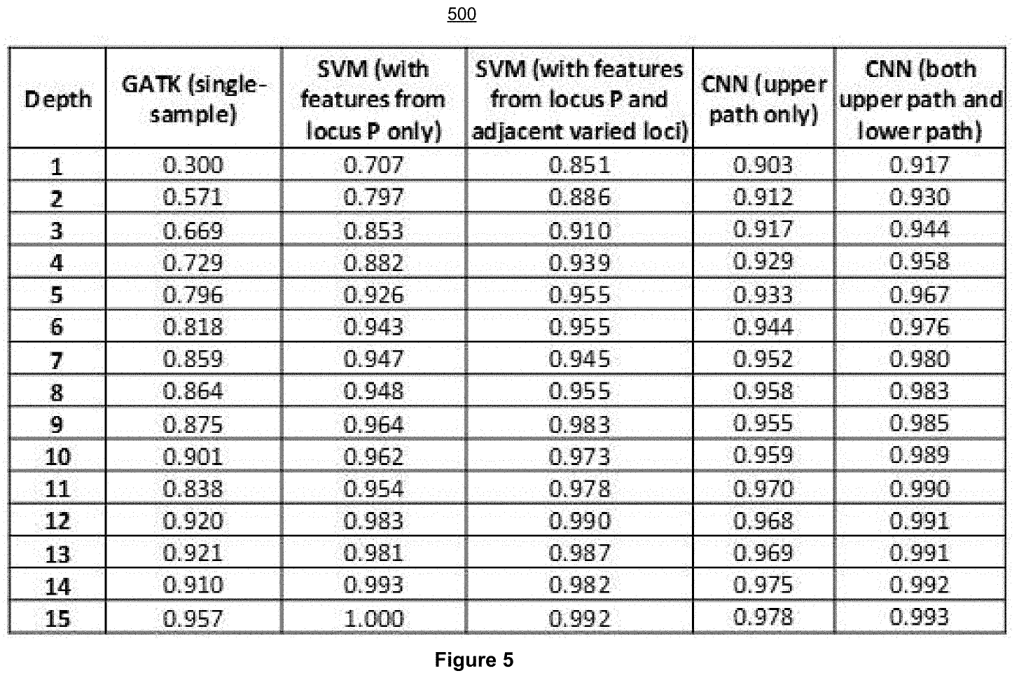

FIG. 5 is a second table 500 illustrating example comparisons of variant calling accuracies achieved using GATK, SVM-based, and CNN-based variant calling methods in accordance with some implementations. FIG. 5 presents the performance of the CNN-based variant caller as compared with GATK and the SVM-based variant caller.

At depth=5, whereas GATK achieves an accuracy of 0.796, the CNN-based variant caller achieves an accuracy of 0.967, even higher than the accuracy achieved by the SVM-based variant caller (0.955). Similarly, at depth=10, the CNN-based variant caller achieves an accuracy of 0.989, in contrast to 0.901 achieved by GATK and 0.973 achieved by the SVM-based variant caller. Notably, the CNN-based variant caller that uses both information from Locus P and information from the varied loci surrounding Locus P (i.e., with the model with both horizontal paths) achieves considerably better performance than the one that uses information from Locus P only (that is, with the model with only the upper path, which is an approximation of DeepVariant). For instance, at depth=5, the former model achieves an accuracy of 0.967, whereas the latter model achieves an accuracy of 0.933. For another instance, at depth=10, the former model achieves an accuracy of 0.989, as compared to an accuracy of 0.959 achieved by the latter model.

Technologies described in the present disclosure was based on varied loci summarized with a population of approximate 200 subjects. With the availability of data from much larger populations (e.g., 100,000 subjects or more), the class 3 variant calling methods described in the present disclosure is expected to offer significantly greater advantage over class 1 and class 2 variant calling methods.

Example Implementations

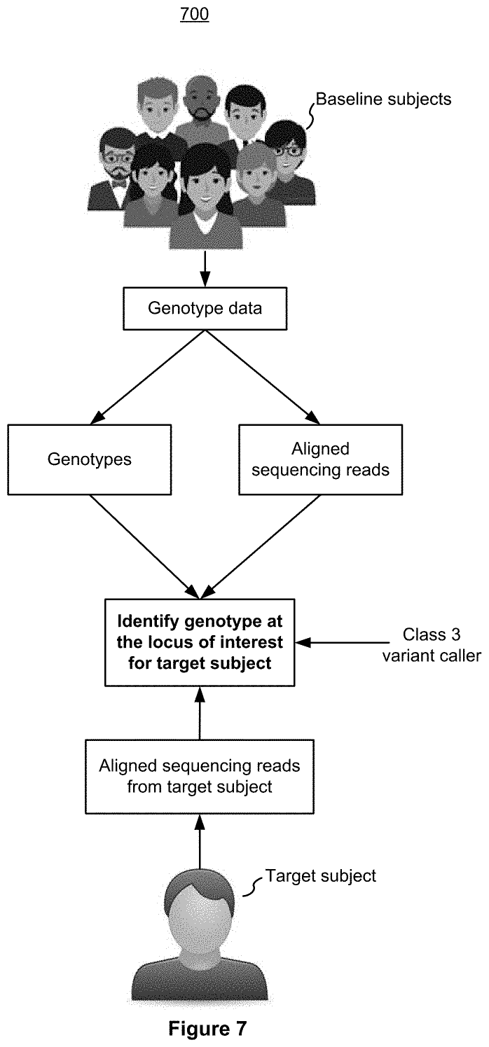

FIG. 7 is an example block diagram illustrating an example method 700 for machine learning-based variant calling in accordance with some implementations. The computer system 900, when programmed properly, can execute the method 700.

In one example method embodiment, a plurality of sequencing reads is generated for a target sequence. Base values at one or more adjacent varied loci of the sequencing reads are used to construe and identify the base value at a locus of interest. In another similar method embodiment of the invention, the base value at a locus of interest is construed and identified by base values at one or more adjacent unchanged loci of the sequencing reads. In an alternative method embodiment of the invention, both base values at one or more adjacent varied and unchanged loci are used to construe and identify the base value at a locus of interest in a target sequence.

In some embodiments of the invention, genotype data are generated from a plurality of baseline subjects, which may have a population of over 200. The correlation of base values among varied loci, such as a percentage number by means of probability values, is established from the genotype data of these baseline subjects. The correlation is then used to apply to the sequencing read of the target sequence and help identify the base value at a locus of interest. In another preferred method embodiment of the invention, the correlation of base values among unchanged loci is established from the genotype data of those baseline subjects. The correlation is applied to the sequencing read of the target sequence to identify the base value at a locus of interest. In a third preferred method embodiment of the invention, the correlation of base values among varied and unchanged loci is established from the genotype data of those baseline subjects before it is applied to the sequencing read of the target sequence to identify the base value at a locus of interest.

In one of the most preferred embodiments of the invention, genotype data are generated from a plurality of baseline subjects. The correlation of base values among varied loci is established from the genotype data of baseline subjects. A plurality of sequencing reads is also produced for a target sequence. Base values at adjacent varied loci of the sequencing reads and correlation values among varied loci in baseline subjects are used to construe and identify the base value at a locus of interest in the target sequence. In another most preferred embodiments of the invention, genotype data are generated from a plurality of baseline subjects. The correlation of base values among unchanged loci is established from the genotype data of baseline subjects. A plurality of sequencing reads is also produced for a target sequence. Base values at adjacent unchanged loci of the sequencing reads and correlation values among unchanged loci in baseline subjects are used to construe and identify the base value at a locus of interest in the target sequence. In a third most preferred embodiment of the invention, base values at adjacent varied and unchanged loci of the sequencing reads and correlation values among varied and unchanged loci in baseline subjects are used to construe and identify the base value at a locus of interest in the target sequence.

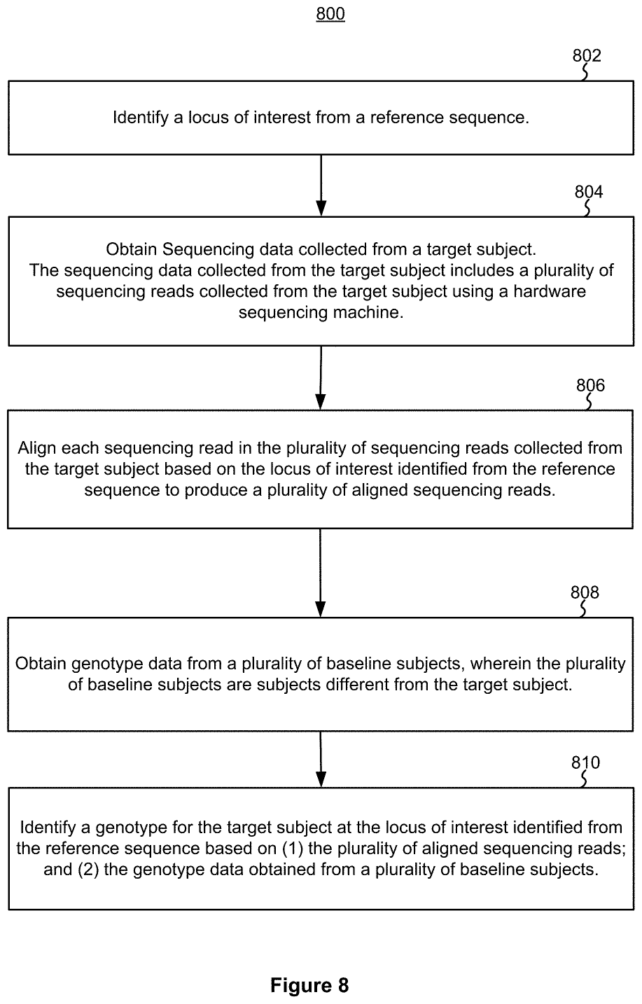

FIG. 8 is an example block diagram illustrating an example method 800 for machine learning-based genotype calling in accordance with some implementations. The computer system 900, when programmed properly, can execute the method 800.

An example computer-implemented genotyping method 800 includes identifying (802) a locus of interest from a reference sequence; and obtaining (804) sequencing data collected from a target subject. The sequencing data collected from the target subject includes a plurality of sequencing reads collected from the target subject using a hardware sequencing machine. The method further includes: aligning (806) each sequencing read in the plurality of sequencing reads collected from the target subject based on the locus of interest identified from the reference sequence to produce a plurality of aligned sequencing reads; obtaining (808) genotype data from a plurality of baseline subjects, and identifying (810) a genotype for the target subject at the locus of interest identified from the reference sequence based on (1) the plurality of aligned sequencing reads and (2) the genotype data obtained from a plurality of baseline subjects. The plurality of baseline subjects are subjects different from the target subject;

Identifying the genotype for the target subject at the locus of interest identified from the reference sequence is, in some implementations, further based on (3) genotype data determined for one or more loci other than the locus of interest for the target subject.

The genotype data includes, in some implementations, one or more genotypes determined for one or more loci for the plurality of baseline subjects.

The one or more loci include, in some implementations, the locus of interest. The one or more loci, in some implementations, excludes the locus of interest. The one or more loci, in some implementations, includes the locus of interest and at least one locus other than the locus of interest.

The genotype data includes, in some implementations, a second plurality of aligned sequencing reads collected from the plurality of baseline subjects.

The method includes, in some implementations, aligning each sequencing read in the second plurality of sequencing reads collected from the baseline subjects based on the locus of interest identified from the reference sequence to produce a second plurality of aligned sequencing reads collected from the baseline subjects.

The aligned sequencing reads of the plurality of baseline subjects include, in some implementations, sequencing reads including the locus of interest and one or more loci other than the locus of interest.

The one or more loci other than the locus of interest include, in some implementations, varied loci that are within a predefined distance from the locus of interest.

The varied loci include, in some implementations, loci with minor allele frequency greater than a predetermined threshold within the plurality of baseline subjects.

The aligned sequencing reads of the plurality of baseline subjects include, in some implementations, sequencing reads that do not include the locus of interest. The aligned sequencing reads of the plurality of baseline subjects, in some implementations, include sequencing reads that include only the locus of interest.

The method includes, in some implementations, identifying the genotype for the target subject at the locus of interest identified from the reference sequence includes applying a machine learning-based classification algorithm. The machine learning-based classification algorithm is trained in accordance with samples with known genotypes.

The classification method used by the machine learning-based classification algorithm is, in some implementations, selected from one of: a Naive Baise (NB) classification method, a Logistic Regression (LR) classification method, a Support Vector Machine (SVM) classification method, and a Random Forest (RF) classification method.

The machine learning-based classification algorithm uses, in some implementations, a classification method selected from one of: a deep fully-connected Artificial Neural Network (ANN) classification method, a Convolutional Neural Network (CNN) classification method, a Recurrent Neural Network (RNN) classification method, or a classification method derived from a network structure derived from any of the preceding network structures.

The hardware sequencing machine is, in some implementations, a next-generation sequencing equipment. The next-generation sequencing equipment, in some implementations, is an Illumina, Ion Torrent, Pacific Biosciences or Oxford Nanopore sequencing machine. The next-generation sequencing equipment, in some implementations, is a high throughput sequencing machine.

The method, in some implementations, is executed by a computer that is different and separate from the hardware sequencing machine. The target subject and the plurality of baseline subjects are, in some implementations, human subjects.

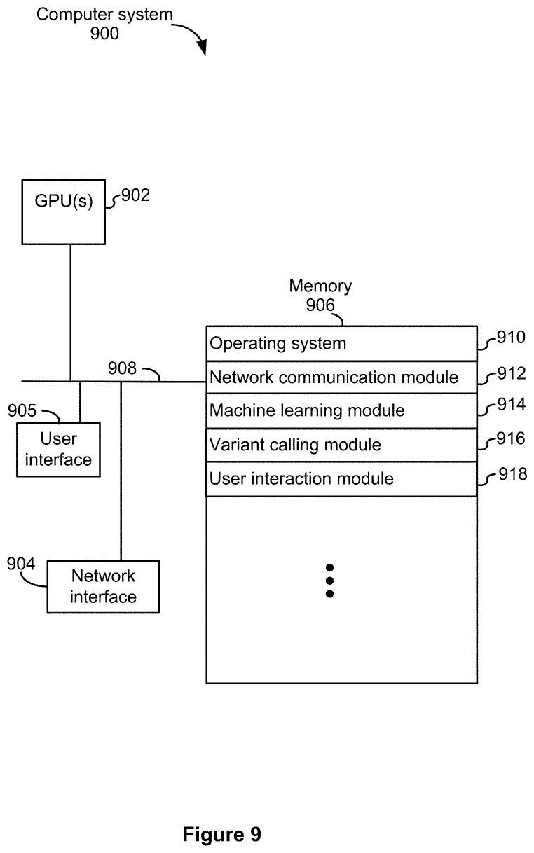

FIG. 9 is an example block diagram illustrating an example computer system 900 for machine learning-based variant calling in accordance with some implementations. The clustering system 900 typically includes one or more Central Processing Units (CPUs) or Graphical Processing Unites (GPUs) 902 (both of which may also be referred to as processors), one or more network interfaces 904, memory 906, and one or more communication buses 908 for interconnecting these components. The communication buses 908 optionally include circuitry (sometimes called a chipset) that interconnects and controls communications between system components. The memory 906 includes high-speed random access memory, such as DRAM, SRAM, DDRRAM or other random access solid state memory devices; and optionally includes non-volatile memory, such as one or more magnetic disk storage devices, optical disk storage devices, flash memory devices, or other non-volatile solid state storage devices. The memory 906 optionally includes one or more storage devices remotely located from GPU(s) 902. The memory 906, or alternatively the non-volatile memory device(s) within the memory 906, comprises a non-transitory computer readable storage medium. In some implementations, the memory 906 or alternatively the non-transitory computer readable storage medium stores the following programs, modules and data structures, or a subset thereof:
an operating system 910 (e.g., an embedded Linux® operating system or a Microsoft Windows® operating system), which includes procedures for handling various basic system services and for performing hardware dependent tasks;
a network communication module 912 for connecting the computer system with a spraying component via one or more network interfaces (wired or wireless);
an machine learning module 914 for providing machine learning related rules and classifications for enabling variant calling;
a variant calling model 916 for determining a genotype, for example, based on machine learning techniques provided by the machine learning module 914; and
a user interaction module 918 for enabling a user to interact with the computer system 900.

In some implementations, one or more of the above identified elements are stored in one or more of the previously mentioned memory devices, and correspond to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 906 optionally stores a subset of the modules and data structures identified above. Furthermore, the memory 906 may store additional modules and data structures not described above.

Although FIG. 9 shows a “computer system 900,” FIG. 9 is intended more as functional description of the various features which may be present in computer systems than as a structural schematic of the implementations described herein. In practice, and as recognized by those of ordinary skill in the art, items shown separately could be combined and some items could be separated.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first camera could be termed a second camera, and, similarly, a second camera could be termed a first camera, without changing the meaning of the description, so long as all occurrences of the “first camera” are renamed consistently and all occurrences of the “second camera” are renamed consistently. The first camera and the second camera are both cameras, but they are not the same camera.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A computer-implemented genotyping method, comprising:
identifying a locus of interest from a reference sequence;
obtaining sequencing data collected from a target subject, wherein the sequencing data collected from the target subject includes a plurality of sequencing reads collected from the target subject using a hardware sequencing machine;
aligning each sequencing read in the plurality of sequencing reads collected from the target subject based on the locus of interest identified from the reference sequence to produce a plurality of aligned sequencing reads;
obtaining genotype data from a plurality of baseline subjects, wherein the plurality of baseline subjects are subjects different from the target subject; and
identifying a genotype for the target subject at the locus of interest identified from the reference sequence based on (1) the plurality of aligned sequencing reads; and (2) the genotype data obtained from a plurality of baseline subjects.

2. The method of claim 1, wherein the genotype data includes one or more genotypes determined for one or more loci for the plurality of baseline subjects.

3. The method of claim 2, wherein the one or more loci include the locus of interest.

4. The method of claim 2, wherein the one or more loci exclude the locus of interest.

5. The method of claim 2, wherein the one or more loci include the locus of interest and at least one locus other than the locus of interest.

6. The method of claim 1, wherein the genotype data includes a second plurality of aligned sequencing reads collected from the plurality of baseline subjects.

7. The method of claim 6, further comprising: aligning each sequencing read in the second plurality of sequencing reads collected from the baseline subjects based on the locus of interest identified from the reference sequence to produce a second plurality of aligned sequencing reads collected from the baseline subjects.

8. The method of claim 6, wherein the aligned sequencing reads of the plurality of baseline subjects include sequencing reads including the locus of interest and one or more loci other than the locus of interest.

9. The method in claim 8, wherein the one or more loci other than the locus of interest include varied loci that are within a predefined distance from the locus of interest.

10. The method of claim 9, wherein the varied loci include loci with minor allele frequency greater than a predetermined threshold within the plurality of baseline subjects.

11. The method of claim 6, wherein the aligned sequencing reads of the plurality of baseline subjects include sequencing reads that do not include the locus of interest.

12. The method of claim 6, wherein the aligned sequencing reads of the plurality of baseline subjects include sequencing reads that include only the locus of interest.

13. The method of claim 1, wherein identifying the genotype for the target subject at the locus of interest identified from the reference sequence includes applying a machine learning-based classification algorithm, wherein the machine learning-based classification algorithm is trained in accordance with samples with known genotypes.

14. The method of claim 13, wherein the machine learning-based classification algorithm uses a classification method is selected from one of: a Naive Baise (NB) classification method, a linear discriminant analysis (LDA) classification method, a Logistic Regression (LR) classification method, a Support Vector Machine (SVM) classification method, and a Random Forest (RF) classification method.

15. The method of claim 14, where a feature selection method is used in conjunction with the classification method.

16. The method of claim 13, wherein the machine learning-based classification algorithm uses a classification method selected from one of: a deep fully-connected Artificial Neural Network (ANN) classification method, a Convolutional Neural Network (CNN) classification method, a Recurrent Neural Network (RNN) classification method, or a classification method derived from a network structure derived from any of the preceding network structures.

17. The method of claim 1, wherein the hardware sequencing machine is a next-generation sequencing equipment and the next-generation sequencing equipment is an Illumina, Ion Torrent, Pacific Biosciences or Oxford Nanopore sequencing machine.

18. The method of claim 16, wherein the next-generation sequencing equipment is a high throughput sequencing machine.

19. The method of claim 1 is executed by a computer that is different and separate from the hardware sequencing machine.

20. The method of claim 1, where identifying the genotype for the target subject at the locus of interest identified from the reference sequence is further based on (3) genotype data determined for one or more loci other than the locus of interest for the target subject.
